(19) United States
(12) Patent Application Publication
(10) Pub. No.: US 2026/0083411 A1
(43) Pub. Date: Mar. 26, 2026

(54) DATA CORRECTION FOR NUCLEAR MEDICINE IMAGING WITH SUPPLEMENTAL TRANSMISSION SOURCE

(71) Applicant: THE BOARD OF REGENTS OF THE UNIVERSITY OF TEXAS SYSTEM, Austin, TX (US)

(72) Inventors: Spencer L. Bowen, Dallas, TX (US); Erik Temesi, Dallas, TX (US)

(21) Appl. No.: 19/111,109

(22) PCT Filed: Sep. 12, 2023
(86) PCT No.: PCT/US2023/073932

§ 371 Date: Mar. 12, 2025

Related U.S. Application Data
(60) Provisional application No. 63/375,664, filed on Sep. 14, 2022.

(51) Int. Cl. A61B 6/03; A61B 6/00

(52) U.S. Cl. CPC A61B 6/037; A61B 6/4417; A61B 6/5205

(57) ABSTRACT

Systems, methods, and devices provide data correction for positron emission tomography (PET) examinations using an external, supplemental radioactive source. The supplemental radioactive source is formed of a uniformly distributed radionuclide. The uniformly distributed radionuclide is positioned in a support structure which is positioned into an inner bore of a PET scanner and secured in place by a friction fit, or directly integrated into the PET system. A transmission source control system moves the one or more 2024/059537 transmission sources into the support structure for performing the PET examination (e.g., using a hydraulic system). The transmission source control system also retracts the one or more transmission sources back into a source storage device upon completing the PET examination. Various data correction algorithms use the first PET signal data, originating from a radiotracer injected in the patient, and second PET signal data, originating from the uniformly distributed radionuclide.

### CROSS-REFERENCE TO RELATED APPLICATION

This application claims priority to U.S. Provisional Patent Application Ser. No. 63/375,664 filed Sep. 14, 2022, titled “DATA CORRECTION FOR NUCLEAR MEDICINE IMAGING WITH SUPPLEMENTAL TRANSMISSION SOURCE,” the entirety of which is incorporated herein by reference.

### ACKNOWLEDGEMENT OF GOVERNMENT SUPPORT

This invention was made with government support under grant number EB028946 awarded by The National Institutes of Health. The government has certain rights in the invention.

### BACKGROUND

### 1. Field

The present inventive concept generally relates to systems and methods to perform a nuclear medicine imaging exam. In at least one example, the present inventive concept relates to a system configured to perform a positron emission tomography (PET) with error correction for signal attenuation.

### 2. Discussion of Related Art

PET is a nuclear medical imaging technique for visualizing and measuring physiological activities inside the body, such as blood flow and metabolic processes. During a typical PET examination, a radioactive substance (e.g., a radiotracer) is injected into a patient, who is then placed inside the bore of a PET scanner. The PET scanner detects and measures the radioactive signal emitted from the radiotracer. However, conventional PET imaging techniques face a variety of issues for generating accurate images that represent radiotracer uptake. Signals from the radiotracer diminish or “attenuate” based on tissue +material properties and their thickness, according to Beer's law.

It is with these observations in mind, among others, that various aspects of the present inventive concept were conceived and developed.

### BRIEF SUMMARY

The presently disclosed technology addresses the foregoing problems by providing systems, methods and devices for nuclear medicine imaging. For example, a nuclear medicine imaging system can include a nuclear medicine scanner having a circular bore for receiving an examination subject; and/or a support structure, positioned into the nuclear medicine scanner, on or near an inner surface of a scanner bore cover. The system can also include one or more external transmission sources configured to be positioned into the support structure; one or more processors; and/or a memory device storing computer-readable instructions that, when executed by the one or more processors, cause the nuclear medicine imaging system to perform operations. The operations can include receiving first imaging signal data associated with a primary source; receiving second imaging signal data associated with the one or more external transmission sources; and/or performing a nuclear medicine signal correction for the first imaging signal data using the first imaging signal data and the second imaging signal data.

In some examples, the nuclear medicine signal correction can be a signal attenuation correction for the first imaging signal data. The support structure can be a support cylinder with a helical channel for receiving the one or more external transmission sources. The nuclear medicine imaging system can also include a source control system which: pushes the one or more external transmission sources into a channel, in the support structure, as part of initiating a nuclear medicine exam procedure; and/or removes the one or more external transmission sources from the channel as part of completing the nuclear medicine exam procedure. The one or more external transmission sources can include a liquid radionuclide; and/or the source control system can include one or more syringes communicatively coupled to one or more pistons for injecting the liquid radionuclide into the helical channel. Additionally, the one or more external transmission sources can include a tube of a radionuclide in a cured epoxy, and/or the source control system can include a storage container for holding a reel of the tube; and/or an automated drive system for moving the tube into and out of the helical channel.

In some scenarios, the primary source can include a radiotracer injected into the examination subject; and/or the one or more external transmission sources can include a uniformly distributed radionuclide. The uniformly distributed radionuclide can be Germanium-68. Additionally, the support structure can include a support cylinder with: a first diameter corresponding to a second diameter of the inner surface of the circular bore; and/or a height dimension corresponding to a length dimension of a scanning component portion at the inner surface of the circular bore. Furthermore, the support structure can be formed of a minimally attenuating material.

In some examples, a method to perform a nuclear medicine imaging examination can include positioning a support structure in a nuclear medicine scanner at an inner surface of a circular bore for receiving an examination subject; positioning one or more external transmission sources into the support structure; receiving first imaging signal data associated with a primary source; receiving second imaging signal data associated with the one or more external transmission sources; and/or performing a nuclear medicine signal correction for the first imaging signal data using the first imaging signal data and the second imaging signal data.

In some examples, the one or more external transmission sources can include a plastic tube filled with a cured epoxy including uniformly distributed Germanium-68. Additionally, the positioning of the support structure in the nuclear medicine scanner can include at least one of using a friction fit to secure the support structure to the inner surface of the circular bore or manufacturing the support structure directly into the nuclear medicine scanner. Moreover, the performing of the nuclear medicine signal correction can include performing a signal attenuation correction for the first imaging signal data. Furthermore, the performing of the nuclear medicine signal correction can include iteratively alternating between generating patient radiotracer image updates, based on the first imaging signal data, and generating attenuation image updates based on the first imaging signal data and the second imaging signal data. By way of example, the generating of the patient radiotracer image updates can use at least an iterative emission image reconstruction algorithm; and/or the generating of the attenuation image updates can use at least an iterative transmission image reconstruction algorithm.

In some scenarios, a nuclear medicine imaging system can include one or more processors; and/or one or more memory devices storing computer-readable instructions that, when executed by the one or more processors, cause the nuclear medicine imaging system perform operations. The operations can include actuating an external source control system causing one or more external transmission sources to be positioned into a support structure, the support structure being positioned or directly integrated in a bore of a PET scanner; receiving first PET signal data associated with a radiotracer administered to a PET examination subject; receiving second PET signal data associated with the one or more external transmission sources; and/or performing a PET signal correction for the first PET signal data using the first PET signal data and the second PET signal data.

In some example, performing the PET signal correction can include segmenting the second PET signal data using a radial threshold; reconstructing, using the second PET signal data, an initial penalized attenuation image with a separable paraboloidal surrogate (SPS) algorithm; reconstructing, using the first PET signal data, an initial patient radiotracer image; generating patient radiotracer image updates using an Ordered Subset Expectation Maximization (OSEM) algorithm; and/or generating attenuation image updates using the SPS algorithm. Furthermore, the nuclear medicine imaging system can include iteratively alternating between updating patient radiotracer images and attenuation images, using both the first PET signal data and the second PET signal data to generate the attenuation images. Additionally, the reconstructing of the initial patient radiotracer image can reduce crosstalk caused by deficient PET detector calibrations.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWING

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system for performing a PET examination using one or more external transmission sources, containing a radionuclide visible on PET, in a support structure;

FIG. 2 illustrates an example system for performing a PET examination using an external transmission source control system, which can form at least a portion of the system depicted in FIG. 1;

FIG. 3 illustrates an example method for performing a PET examination using one or more external transmission sources, which can be performed by the system depicted in FIG. 1; and

FIG. 4 illustrates an example method for performing a PET examination using one or more external transmission sources, which can be performed by the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present inventive concept.

### I. TERMINOLOGY

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present inventive concept be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present inventive concept, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

### II. GENERAL ARCHITECTURE

The systems, methods, and devices disclosed herein improve quantification of nuclear medicine images produced, for instance, from Positron Emission Tomography (PET) systems through the addition of a supplemental radioactive source. This disclosed technology corrects for the primary source of bias in nuclear medicine images (e.g., PET images)—attenuation of the imaging signal. The system can perform direct measurement and correction for the attenuation effect using imaging data from the emission source(s) alone without requiring image data from other cameras, prior assumptions, or training data. This improves many use cases where attenuation information is unavailable (e.g. on a standalone brain PET system) or unreliable (e.g. on a combined PET and MRI scanner), or correction for attenuation impacts patient safety (e.g. due to X-ray computed tomography (CT) radiation dose in a combined PET and CT scanner).

In some examples, the system includes a device that can place and remove an external positron-emitting source (e.g., visible on PET) without impacting clinical operations. The system can also include a scheme for using a PET signal from either the external source alone (e.g., a “transmission” signal from one or more external transmission sources), or combined with that originating from the patient, to measure and correct for PET attenuation. The system can be used with or include various types of standalone PET and hybrid PET/CT and PET/Magnetic Resonance (MR) scanners.

In some scenarios, the disclosed technology can be integrated into a PET scanner, such as a standalone PET, or hybrid PET/MR and PET/CT scanners without substantial effort from the technologists, without negatively impacting PET image quality, and without added radiation safety concerns. For instance, the external transmission source(s) can be automatically placed and removed. Moreover, the techniques can omit any requirement to move the patient table to estimate patient attenuation, and can operate after the patient has been injected with radiotracer. Additionally, it is to be understood that the technology disclosed herein can be used with other types of nuclear medicine scanners, such as a standalone PET system, a combined PET and magnetic resonance imaging (MRI) scanner (PET/MR), a hybrid PET and CT scanner (PET/CT), a Single Photon Emission Computed Tomography (SPECT) scanner (either alone or combined with a CT scanner), and so forth.

Additional advantages of the systems, methods, and devices discussed herein will become apparent from the detailed description below.

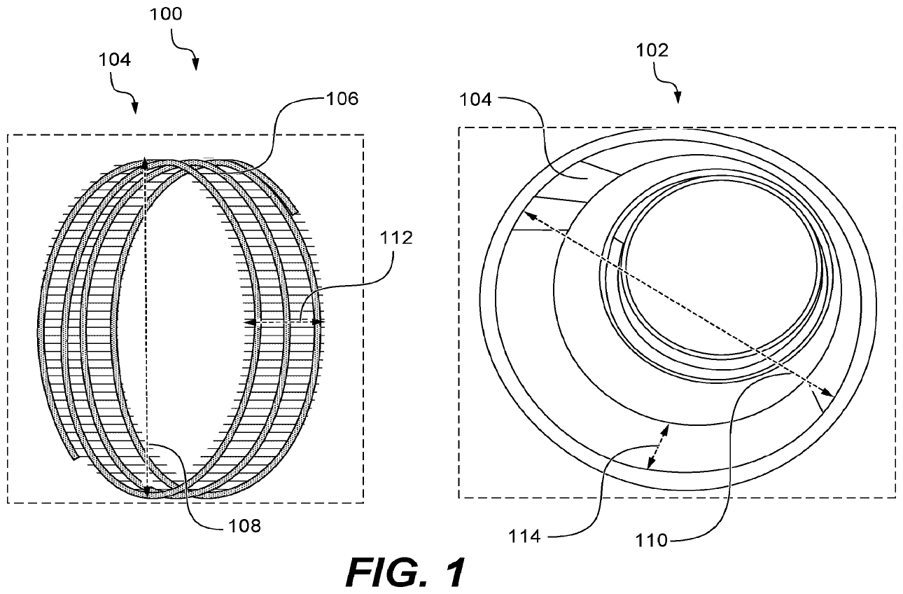

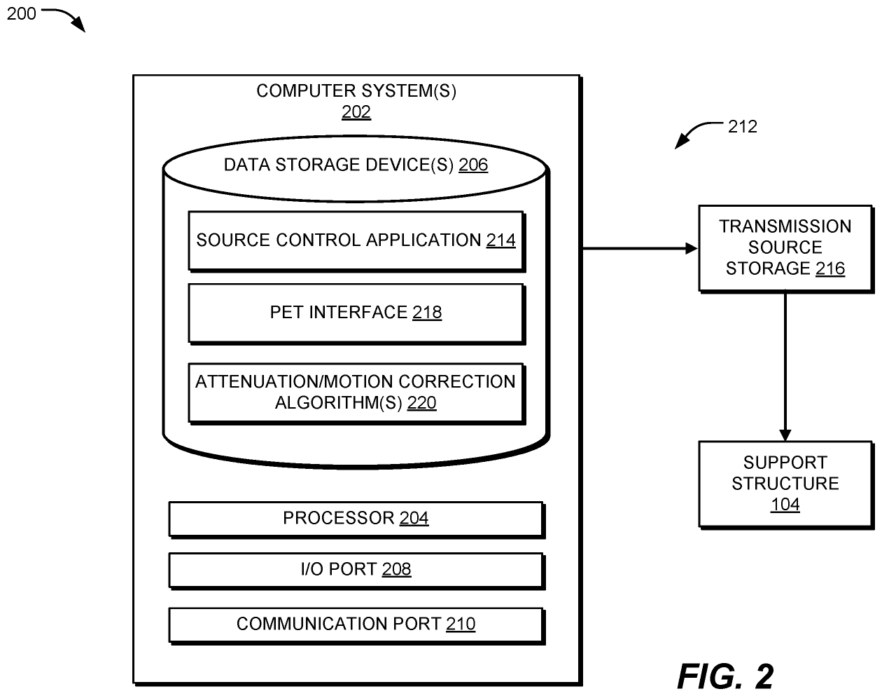

FIG. 1 illustrates an example PET system 100 including a PET scanner 102 with a circular bore for receiving a PET examination subject (e.g., a patient). A support structure 104, which can be formed of a minimally attenuating material such as polycarbonate, can be positioned in the circular bore for holding the external transmission sources in place. In some instances, the support structure 104 is a support cylinder, a support strip, a support ring, or other type of support structure shaped to fit in the bore of the PET scanner 102. One or more external transmission sources 106 can be positioned in the support structure 104, such as in one or more channels formed into the support structure 104. The external transmission source(s) 106 can be a line source, a point source, a ribbon-shaped source, other source shapes, combinations thereof, and so forth. Additionally, in some instances (e.g., where the support structure 104 is a support cylinder), the one or more channels can be one or more helical channels. The support structure may be directly integrated into the patient bore cover (e.g., manufactured into the patient bore cover), or placed into the patient bore cover in a removable/interchangeable manner (e.g., by personnel operating the PET scanner 102). Furthermore, the PET system 100 can include a source control system for moving the external transmission source(s) 106 into and out of the support structure 104, as discussed below regarding FIG. 2.

In some examples a PET signal correction can be performed using the external transmission source(s) 106, which can be placed in a sparse configuration near or in the bore cover of the PET scanner 102. The PET signal correction can be a signal attenuation correction applied to PET signal data originating from the radiotracer injected into the patient. The PET signal correction can use various image reconstruction algorithms, such as an Ordered Subset Expectation Maximization (OSEM) algorithm and/or a Separable Paraboloidal surrogate (SPS) algorithm, as discussed in greater detail below.

In some instances, the PET system 100 uses first PET signal data from a primary source (e.g., the radiotracer injected into the patient) and second PET signal data from a second source, such as the external transmission source(s) 106. The external transmission source(s) 106 can include a uniformly distributed radionuclide, such as Germanium-68, which can be in an injectable liquid form in a tube (e.g., a Teflon tube) or cured epoxy.

Additionally, in some scenarios, the support structure 104 has a first diameter 108 corresponding to a second diameter 110 of the inner surface of the circular bore (e.g., having approximately a same dimension or nearly a same dimension within an error range). As such, the support structure 104 can be positioned in the PET scanner 102 using a friction fit that secures the support structure 104 against the inner surface of the circular bore. Furthermore, the support structure 104 can have a height dimension 112 that corresponds to a length dimension 114 of the inner surface of the circular bore. In some instances the height dimension 112 can be a same dimension as the length dimension 114, or the height dimension 112 can be proportional to the length dimension 114 (e.g., half of the length dimension 114) and/or less than the length dimension 114. The height dimension 112 can correspond to a length dimension 114 that is a portion of the circular bore including the scanning sensors of the PET scanner 102

FIG. 2 illustrates an example computing architecture 200 which may form at least a portion of the system 100 discussed herein. Referring to FIG. 2, the computing architecture 200 can include an example computer system 202 having one or more computing units which may implement the systems and methods discussed herein. The computer system 202 can communicate with the PET scanner 102 and/or be integrated with the PET scanner 102. It will be appreciated that specific implementations of these devices may be of differing possible specific computing architectures not all of which are specifically discussed herein but will be understood by those of ordinary skill in the art.

The computer system 202 may be capable of executing a computer program product and/or a computer process to perform the operations discussed herein. Data and program files may be input to the computer system 202, which reads the files and executes the programs therein. Some of the elements of the computer system 202 are shown in FIG. 2 and include one or more hardware processors 204, one or more data storage devices 206, one or more I/O ports 208, and/or one or more communication ports 210. Various elements of the computer system 202 may communicate with one another by way of one or more communication buses, point-to-point communication paths, or other communication means. Furthermore, the computer system 202 can include a source control system 212 which can include at least a source control application 214 (e.g., software) stored at the device(s) 206. The source control application 214 can communicate with and/or control a transmission source storage 216 that stores the transmission source(s) 106. Furthermore, the computer system 202 can include a PET interface 218. Additionally or alternatively, the source control system 212 can include or communicate with a hydraulic drive system to automatically place and remove the external transmission source(s) 106, as discussed in greater detail below.

In some instances, the computer system(s) 202 may be a computer, a desktop computer, a laptop computer, a cellular or mobile device, a smart mobile device, a wearable device (e.g., a smart watch, smart glasses, a smart epidermal device, etc.) an Internet-of-Things (IoT) device, a smart home device, a virtual reality (VR) or augmented reality (AR) device, combinations thereof, and the like. The computer system 202 can provide operational control over the PET system 100. For instance, the PET interface 218 can provide data commands and/or API calls to and from the PET scanner 104 so that operations of the computer system 202 (e.g., actuating the source control system 212) are responsive to operations of the PET scanner 104 (e.g., initiating or completing a PET examination). The computer system 202 may be a standalone computer a distributed computer, multiple intercommunicating computers, or any other type of computer, such as one or more external computers made available via a cloud computing architecture. The presently described technology is optionally implemented in software stored on the data storage device(s) 206 and/or communicated via the one or more of the I/O port(s) 208 and/or communication port(s) 210, thereby transforming the computer system 202 in FIG. 2 to a special purpose machine for implementing the PET system 100.

The processor 204 may include, for example, a central processing unit (CPU), a microprocessor, a microcontroller, a digital signal processor (DSP), and/or one or more internal levels of cache. There may be one or more processors 204, such that the processor 204 comprises a single central-processing unit, or a plurality of processing units capable of executing instructions and performing operations in parallel with each other, commonly referred to as a parallel processing environment.

The one or more data storage device(s) 206 may include any non-volatile data storage device capable of storing data generated or employed within the computer system 202, such as computer-executable instructions for performing a computer process, which may include instructions of both application programs and an operating system (OS) that manages the various components of the computer system 202. The data storage device(s) 206 may include, without limitation, magnetic disk drives, optical disk drives, solid state drives (SSDs), flash drives, and the like. The data storage devices 206 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The data storage device(s) 206 may include volatile memory (e.g., dynamic random-access memory (DRAM), static random access memory (SRAM), etc.) and/or non-volatile memory (e.g., read-only memory (ROM), flash memory, etc.). The data storage device may include a non-transitory machine-readable medium having stored thereon instructions, which may be used to program a computer system (or other electronic devices) to perform a process according to the present inventive concept. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

Computer program products containing mechanisms to effectuate the systems and methods in accordance with the presently described technology may reside in the data storage device(s) 206, which may be referred to as machine-readable media. It will be appreciated that machine-readable media may include any tangible non-transitory medium that is capable of storing or encoding instructions to perform any one or more of the operations of the present inventive concept for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures. The machine-readable media may store instructions that, when executed by the processor, cause the systems to perform the operations disclosed herein.

For instance, the device(s) 206 can store the source control application 214, the PET interface 218, and/or one or more attenuation/motion correction algorithms 220. The source control application 214 can be algorithmic components to actuate the source control system 212 (e.g., a hydraulic piston) to cause the external transmission source(s) 106 to be pushed into the helical channel(s) in the support structure 104, for instance, to initiate or, as part of the imitation, for a PET examination procedure (e.g., responsive to a user input and/or an initiation signal from the PET scanner 102). Additionally or alternatively, the source control application 214 can acuate the source control system 212 to remove the external transmission source(s) 106 from the helical channel(s), for instance, to complete, or as part of completing, the PET examination procedure. In some scenarios, the external transmission source(s) 106 are a liquid radionuclide and the transmission source storage 216 includes one or more syringes holding the liquid radionuclide. Actuating the source control system 212 can cause the one or more syringes communicatively coupled to one or more pistons to inject the liquid radionuclide into the helical channels. Additionally or alternatively, the external transmission source(s) 106 include the radionuclide dispersed in a cured epoxy in a tube and the transmission source storage 216 can include a reel around which the tube is wrapped. The transmission source storage 216 can also include a radiation shielded container (e.g. lead box) for holding the external transmission source(s) 106. Upon actuating the source control system 212, the hydraulic drive system can unwind the wrapped tube of radionuclide from the reel and push the tube into the helical channel(s). Once the PET examination procedure is complete, the hydraulic drive system can operate in reverse to rewind the tube back onto the real in the lead box.

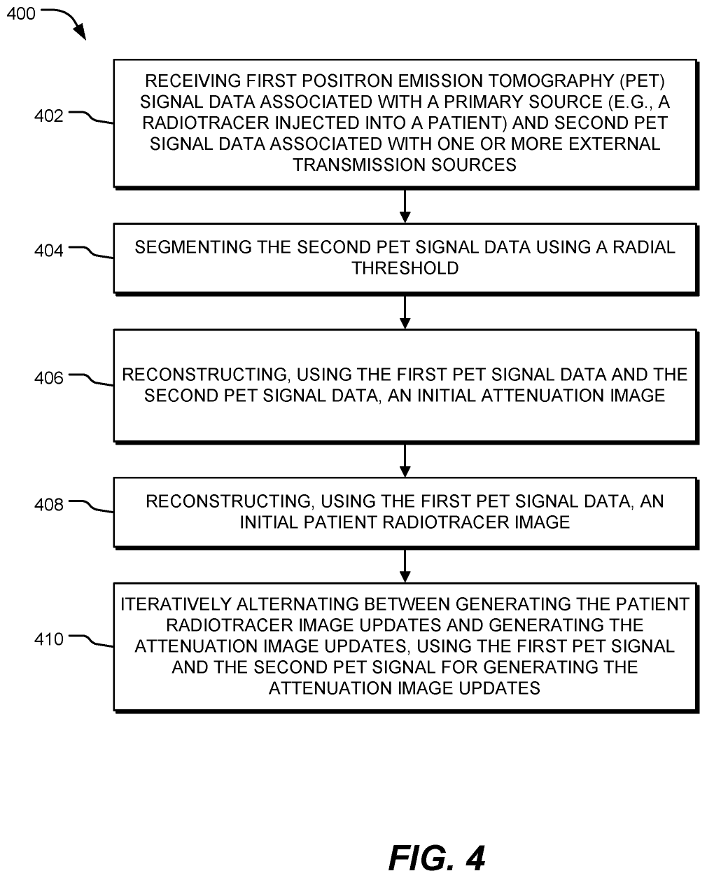

Furthermore, the source control system 212 can implement the one or more attenuation/motion correction algorithms 220. In some instances, the attenuation/motion correction algorithms 220 include a joint uptake reconstruction-registration (JRM) algorithm using an alternating update strategy. For a motion estimation scheme to correct for patient motion, a penalized maximum-likelihood optimization can be implemented by employing a Broyden-Fletcher-Goldfarb-Shanno (BFGS) algorithm on the first PET signal data and/or second PET signal data. The penalized log-likelihood (LL) can contain the log-likelihood of the noisy transmission measurement from the external transmission source(s) 106 combined with a weak Cauchy prior (e.g., penalizing sudden changes in transform parameters between neighboring frames). The BFGS can be run to convergence using a line search with Strong Wolfe Conditions. An attenuation map update for correcting for signal attenuation can utilize an estimated rigid body transformation in a motion-compensated separable paraboloidal surrogate (SPS) algorithm and can be run for, in some instances, 20 iterations. Additional examples of the attenuation/motion correction algorithms 220 and operations are discussed in greater detail below regarding FIG. 4.

In some implementations, the computer system 202 includes one or more ports, such as the one or more input/output (I/O) port(s) 208 and the one or more communication port(s) 210, for communicating with other computing devices, network devices, the source control system 212, the PET scanner 102, or combinations thereof. It will be appreciated that the I/O port(s) 208 and the communication port(s) 210 may be combined or separate and that more or fewer ports may be included in the computer system 202.

The I/O port(s) 208 may be connected to an I/O device, or other device, by which information is input to or output from the computer system 202. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computer system 202 via the I/O port 208. Similarly, the output devices may convert electrical signals received from computer system 202 via the I/O port 208 into signals that may be sensed as output by a human, such as sound, light, and/or touch. The input device may be an alphanumeric input device, including alphanumeric and other keys for communicating information and/or command selections to the processor 204 via the I/O port 208. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”) with a graphical user interface (GUI). The output devices may include, without limitation, a display, a touchscreen, a projector, a speaker, a tactile and/or haptic output device, and/or the like. In some implementations, the input device and the output device may be the same device, for example, in the case of a touchscreen.

In one implementation, a communication port 210 is connected to the various components of the PET system 100, such as the PET scanner 102 and/or the source control system 212. The communication port 210 can connect through over a local area network (LAN), a wide area network (WAN) (e.g., the Internet), one or more scientific equipment application programming interface (API) connections or other communication protocols. The communication port 210 can provide various other types of connections, such as for a Universal Serial Bus (USB), Ethernet, Wi-Fi, Bluetooth®, Near Field Communication (NFC), a cellular network (e.g., a Third Generation Partnership Program (3GPP) network), and the like. Further, the communication port 210 may communicate with an antenna or other link for electromagnetic signal transmission and/or reception.

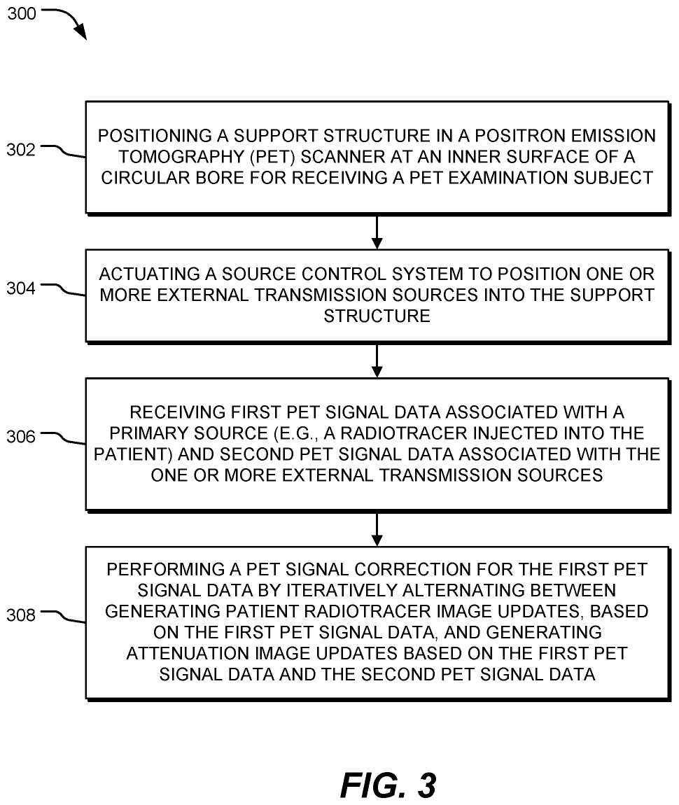

In an example implementation, operations performed by the systems discussed herein may be embodied by instructions stored on the data storage devices 206 and executed by the processor 204, for instance, using the source control application 214, the PET interface 218, and/or the attenuation/motion correction algorithm(s) 220. The computer system 202 set forth in FIG. 2 is but one possible example of a computer system that may be employed or configured in accordance with aspects of the present inventive concept. It will be appreciated that other non-transitory tangible computer-readable storage media storing computer-executable instructions for implementing the presently disclosed technology on a computing system may be utilized. The methods disclosed herein, such as methods 300 and 400 depicted in FIGS. 3 and 4, may be implemented as sets of instructions or software readable by the computer system 202.

FIG. 3 illustrates a flow chart of an example method 300 for performing an attenuation correction and/or a patient motion correction for a PET examination, which can be performed by the PET system 100 and/or the computing architecture 200.

In some instances, at operation 302, the method 300 positions a support structure in a PET scanner at an inner surface of a circular bore for receiving a PET examination subject. At operation 304, the method 300 actuates a source control system to position one or more external transmission sources into the support structure. At operation 306, the method 300 receives first PET signal data associated with a primary source (e.g., a radiotracer injected into the patient) and second PET signal data associated with the one or more external transmission sources. At operation 308, the method 300 performs a PET signal correction for the first PET signal data by iteratively alternating between generating patient radiotracer image updates, based on the first PET signal data, and generating attenuation image updates based on the first PET signal data and the second PET signal data. In some instances, operation 308 includes executing one or more machine-learning algorithms to perform an image reconstruction procedure on the first PET signal data by using the second PET signal data, such as deep learning, supervised learning, unsupervised learning, regressions, neural networks, decision trees, gradient boosting, and the like.

FIG. 4 illustrates a flow chart of an example method 400 for performing an attenuation correction and/or a motion correction for a PET examination, which can be performed by the PET system 100 and/or the computing architecture 200.

In some examples, at operation 402, the method 400 receives first PET signal data associated with a primary source (e.g., a radiotracer injected into the patient) and second PET signal data associated with one or more external transmission sources. At operation 404, the method 400 segments the second PET signal data using a radial threshold. At operation 406, the method 400 reconstructs, using the first PET signal data and the second PET signal data, an initial attenuation image. Operation 406 can further include reconstructing a penalized maximum-likelihood attenuation image with a separable paraboloidal surrogate (SPS) algorithm. At operation 408, the method 400 reconstructs, using the first PET signal data, an initial patient radiotracer image. At operation 410, the method 400 iteratively alternates between generating the patient radiotracer image updates and generating the attenuation image updates, using the first PET signal and the second PET signal for generating the attenuation image updates.

It is to be understood that the specific order or hierarchy of steps in the method depicted in FIGS. 3 and 4 and throughout this disclosure are instances of example approaches and can be rearranged while remaining within the disclosed subject matter. For instance, any of the operations depicted in FIGS. 3 and 4 and throughout this disclosure can be omitted, repeated, performed in parallel, performed in a different order, and/or combined with any other of the operations depicted in FIGS. 3 and 4 and throughout this disclosure. Moreover, any of the systems or methods illustrated in FIGS. 1-4 can be combined together and/or form at least a portion of the PET system 100.

While the present inventive concept has been described with reference to various implementations, it will be understood that these implementations are illustrative and that the scope of the present inventive concept is not limited to them. Many variations, modifications, additions, and improvements are possible. More generally, implementations in accordance with the present inventive concept have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A nuclear medicine imaging system comprising:
a nuclear medicine scanner having a circular bore for receiving an examination subject;
a support structure, positioned into the nuclear medicine scanner, on or near an inner surface of a scanner bore cover;
one or more external transmission sources configured to be positioned into the support structure;
one or more processors; and
a memory device storing computer-readable instructions that, when executed by the one or more processors, cause the nuclear medicine imaging system to:
receive first imaging signal data associated with a primary source;
receive second imaging signal data associated with the one or more external transmission sources; and
perform a nuclear medicine signal correction for the first imaging signal data using the first imaging signal data and the second imaging signal data.

2. The nuclear medicine imaging system of claim 1,
wherein,
the nuclear medicine signal correction is a signal attenuation correction for the first imaging signal data.

3. The nuclear medicine imaging system of claim 1,
wherein,
the support structure is a support cylinder with a helical channel for receiving the one or more external transmission sources.

4. The nuclear medicine imaging system of claim 3, further comprising:
a source control system which:
pushes the one or more external transmission sources into a channel, in the support structure, as part of initiating a nuclear medicine exam procedure; and
removes the one or more external transmission sources from the channel as part of completing the nuclear medicine exam procedure.

5. The nuclear medicine imaging system of claim 4,
wherein,
the one or more external transmission sources include a liquid radionuclide; and
the source control system includes one or more syringes communicatively coupled to one or more pistons for injecting the liquid radionuclide into the helical channel.

6. The nuclear medicine imaging system of claim 4,
wherein,
the one or more external transmission sources include a tube of a radionuclide in a cured epoxy, and
the source control system includes:
a storage container for holding a reel of the tube; and
an automated drive system for moving the tube into and out of the helical channel.

7. The nuclear medicine imaging system of claim 1,
wherein,
the primary source includes a radiotracer administered to the examination subject; and
the one or more external transmission sources include a uniformly distributed radionuclide.

8. The nuclear medicine imaging system of claim 7,
wherein,
the uniformly distributed radionuclide is Germanium-68 or any positron-emitting radionuclide.

9. The nuclear medicine imaging system of claim 1,
wherein,
the support structure includes a support cylinder with:
a first diameter corresponding to a second diameter of the inner surface of the circular bore; and
a height dimension corresponding to a length dimension of a scanning component portion at the inner surface of the circular bore.

10. The nuclear medicine imaging system of claim 1,
wherein,
the support structure is formed of a minimally attenuating material.

11. A method to perform a nuclear medicine imaging examination, the method comprising:
positioning a support structure in a nuclear medicine scanner at an inner surface of a circular bore for receiving an examination subject;
positioning one or more external transmission sources into the support structure;
receiving first imaging signal data associated with a primary source;
receiving second imaging signal data associated with the one or more external transmission sources; and
performing a nuclear medicine signal correction for the first imaging signal data using the first imaging signal data and the second imaging signal data.

12. The method of claim 11,
wherein,
the one or more external transmission sources includes a plastic tube filled with a cured epoxy including uniformly distributed Germanium-68, or any positron-emitting radionuclide.

13. The method of claim 11,
wherein,
the positioning of the support structure in the nuclear medicine scanner includes at least one of using a friction fit to secure the support structure to the inner surface of the circular bore or manufacturing the support structure directly into the nuclear medicine scanner.

14. The method of claim 11,
wherein,
the performing of the nuclear medicine signal correction includes performing a signal attenuation correction for the first imaging signal data.

15. The method of claim 11,
wherein,
the performing of the nuclear medicine signal correction includes iteratively alternating between generating patient radiotracer image updates, based on the first imaging signal data, and generating attenuation image updates based on the first imaging signal data and the second imaging signal data.

16. The method of claim 15,
wherein,
the generating of the patient radiotracer image updates uses at least an iterative emission image reconstruction algorithm; and
the generating of the attenuation image updates uses at least an iterative transmission image reconstruction algorithm.

17. A nuclear medicine imaging system comprising:
one or more processors; and
one or more memory devices storing computer-readable instructions that, when executed by the one or more processors, cause the nuclear medicine imaging system to:
actuate an external source control system causing one or more external transmission sources to be positioned into a support structure, the support structure being positioned or directly integrated in a bore of a PET scanner;
receive first PET signal data associated with a radiotracer injected into a PET examination subject;
receive second PET signal data associated with the one or more external transmission sources; and
perform a PET signal correction for the first PET signal data using the first PET signal data and the second PET signal data.

18. The nuclear medicine imaging system of claim 17,
wherein,
performing the PET signal correction includes:
segmenting the second PET signal data using a radial threshold;
reconstructing, using the second PET signal data, an initial penalized attenuation image with a separable paraboloidal surrogate (SPS) algorithm;
reconstructing, using the first PET signal data, an initial patient radiotracer image;
generating patient radiotracer image updates using an Ordered Subset Expectation Maximization (OSEM) algorithm; and
generating attenuation image updates using the SPS algorithm.

19. The nuclear medicine imaging system of claim 18, further comprising:
iteratively alternating between updating patient radiotracer images and attenuation images, using both the first PET signal data and the second PET signal data to generate the attenuation images. CM 20. The nuclear medicine imaging system of claim 18,
wherein,
the reconstructing of the initial patient radiotracer image reduces crosstalk caused by deficient PET detector calibrations.
